(19) United States
(12) Patent Application Publication
(10) Pub. No.: US 2023/0344907 A1
(43) Pub. Date: Oct. 26, 2023

(54) Systems and Methods for Techniques to Process, Manage, and Use Neural Signal Data

(71) Applicant: The Trustees of Columbia University in the City of New York, New York, NY (US)

(72) Inventor: Baihan Lin, New York, NY (US)

(21) Appl. No.: 18/137,734

(22) Filed: Apr. 21, 2023

Related U.S. Application Data
(60) Provisional application No. 63/389,096, filed on Jul. 14, 2022.
(60) Provisional application No. 63/333,791, filed on Apr. 22, 2022.

(51) Int. Cl. H04L 67/133; G06F 16/25; G06F 9/54; G06N 3/092; G06F 3/01

(52) U.S. Cl. CPC H04L 67/133; G06F 16/258; G06F 9/547; G06F 9/546; G06N 3/092; G06F 3/015

(57) ABSTRACT

Disclosed are methods, systems, and other implementations for processing and managing high volume complex data (such as captured neural signals data). The implementations include a method that includes receiving at a first network device, from a remote, second network device, a remote procedure call (RPC) message comprising a first data representation of neural signal data obtained by the second network device and servicing data specifying parameters to cause execution of a first servicing procedure executable on the first network device, performing the first servicing procedure to process the first data representation of the neural signal data to generate result data, and transmitting, by the first remote network device, another RPC message to a destination network device, the other RPC message including the result data.

### CROSS-REFERENCE TO RELATED APPLICATIONS

This application claims the benefit of, and priority to, U.S. Provisional Application No. 63/333,791, entitled “Systems and Methods for Serialization, Deserialization and Storage of Neural Data With Protocol Buffers,” and filed Apr. 22, 2022, and to U.S. Provisional Application No. 63/389,096, entitled “Systems and Methods for Prediction and Recommendation Operations Based on Neural Signals in Social Setting” and filed Jul. 14, 2022, the contents of all of which are incorporated herein by reference in their entireties.

### BACKGROUND

With increasing complexity and magnitude of neural data arising from recent high-throughput multi-channel neurophysiology and neuroimaging techniques, the standardization of data storage, data communication, and data processing are important elements to promote reproducibility and collaboration in neuroscience. Although projects like Neurodata-Without-Borders initiative has made progress in the way of data standardization in neuroscience, there are still several outstanding challenges: (1) efficient storage and fast retrieval are at an increasingly irreconcilable tradeoff given the increasingly combinatorial numbers of meta information; (2) high-dimensionality of spatial and temporal resolutions from single cell and multi-channel techniques prevents a workable analytical pipeline in local machines and hard disks; (3) analytical pipelines are adopting resource-heavy models like deep learning which pose additional bandwidth and computational constraints. This is especially the case in cognitive neuroscience, where the recordings of neural responses often accompany high-dimensional stimulus inputs, hierarchical meta information, and delicate cognitive model architectures for neurobiological inference.

### SUMMARY

Disclosed are implementations (including hardware, software, and hybrid hardware/software implementations) directed to several frameworks and techniques for processing and managing voluminous complex data (such as captured neural signals data). An example application, namely, a recommendation platform to make recommendations based on collected neural signal data from multiple individuals, that uses the data management frameworks and techniques presented herein, is also described.

Thus, in some variations, a first method, for management of neural data, is provided that includes obtaining one or more samples of neural data, and processing the one or more samples of neural data according to protocol buffer definitions specifying formatting of neural data records for storage and transmission, to generate formatted neural data records.

In some variations, a first system, for data management system, is provided that includes one or more memory devices to store processor-executable instructions and neural data, and a processor-based controller, coupled to the one or more memory devices. The controller is configured, when executing the processor-executable instructions, to obtain one or more samples of neural data, and process the one or more samples of neural data according to protocol buffer definitions specifying formatting of neural data records for storage and transmission, to generate formatted neural data records.

In some embodiments, examples, a first non-transitory computer readable media is provided that includes computer instructions executable on a processor-based device to obtain one or more samples of neural data, and process the one or more samples of neural data according to protocol buffer definitions specifying formatting of neural data records for storage and transmission, to generate formatted neural data records.

In some variations, a second method, for processing and communicating neural signal data, is provided. The method includes receiving at a first network device, from a remote, second network device, a remote procedure call (RPC) message comprising a first data representation of neural signal data obtained by the second network device and servicing data specifying parameters to cause execution of a first servicing procedure executable on the first network device, performing the first servicing procedure to process the first data representation of the neural signal data to generate result data, and transmitting, by the first remote network device, another RPC message to a destination network device, the other RPC message including the result data.

In some embodiments, a second system, for neurotech communication, is provided that includes multiple network devices comprising at least a first network device and a second network device (e.g., a neurotech device that collects neural signals), with each of the multiple network devices including one or more memory devices to store processor-executable instructions and neural signal data, and a processor-based controller coupled to the one or more memory devices. The processor-based controller of the first network device is configured, when executing associated processor-executable instructions, to receive at the first network device, from the second network device, a remote procedure call (RPC) message comprising a first data representation of neural signal data obtained by the second network device and servicing data specifying parameters to cause execution of a first servicing procedure executable on the first network device, perform the first servicing procedure to process the first data representation of the neural signal data to generate result data, and transmit, by the first remote network device, another RPC message to a destination network device, the other RPC message including the result data.

In some embodiments, a second non-transitory computer readable media is provided that includes computer instructions executable on one or more processor-based devices to receive at a first network device, from a remote, second network device, a remote procedure call (RPC) message comprising a first data representation of neural signal data obtained by the second network device and servicing data specifying parameters to cause execution of a first servicing procedure executable on the first network device, perform the first servicing procedure to process the first data representation of the neural signal data to generate result data, and transmit, by the first remote network device, another RPC message to a destination network device, the other RPC message including the result data.

In some variations, a third method is provided that includes obtaining from multiple users neural signals relating to an item, obtaining a pre-determined user rating for the item, deriving a collective neural-signal-based rating for the item based on the pre-determined user rating and the neural signals from the multiple users, and performing an item-related operation based on the collective neural-signal-based rating for the item.

In some embodiments, a third system is provided that includes multiple brain-computer interface devices to obtain from multiple users neural signals relating to an item, and one or more processor-based controllers, in communication with the brain-computer interface devices. The one or more processor-based controllers are configured to obtain a pre-determined user rating for the item, derive a collective neural-signal-based rating for the item based on the pre-determined user rating and the neural signals from the multiple users, and perform an item-related operation based on the collective neural-signal-based rating for the item.

In some embodiments, a third non-transitory computer readable media is provided that includes computer instructions executable on one or more processor-based devices to obtain from multiple users neural signals relating to an item, obtain a pre-determined user rating for the item, derive a collective neural-signal-based rating for the item based on the pre-determined user rating and the neural signals from the multiple users, and perform an item-related operation based on the collective neural-signal-based rating for the item.

Embodiments and variations of any of first, second, and third methods, systems, and computer readable media may include at least some of the features described in the present disclosure, including at least some of the features described above in relation to the methods, the systems, and the computer-readable media. Furthermore, any of the above variations and embodiments of the methods, systems, and/or computer-readable media, may be combined with any of the features of any other of the variations of the methods, systems, and computer-readable media described herein, and may also be combined with any other of the features described herein.

Other features and advantages of the invention are apparent from the following description, and from the claims.

### BRIEF DESCRIPTION OF THE DRAWINGS

These and other aspects will now be described in detail with reference to the following drawings.

FIG. 1 is a diagram of an example processing pipeline to process data (such as neural data).

FIG. 2 includes an example Xneuro proto file.

FIG. 3 includes another example Xneuro proto file.

FIG. 4 is a flowchart of an example procedure for managing and processing neural data.

FIG. 5A includes graphs showing the time (ms) required to respectively save and load a neural signal of various lengths, for different storage types.

FIG. 5B includes a graph showing file size (bytes) required to save a neural signal of various lengths for different storage types.

FIG. 6 includes a table summarizing the performance comparison results of the Xneuro framework against other tested industry solutions.

FIG. 7 is a flow diagram depicting an example messaging procedure to trigger/invoke a remote neurotech procedure.

FIG. 8 is a schematic diagram of an example neurotech RPC-based network.

FIG. 9 is a diagram of an example gPRC-based network.

FIG. 10 includes screenshots of a working example of RPC-based communication between a client and a server in a neurotech network.

FIG. 11 is a flowchart of an example procedure for processing and communicating neural signal data.

FIG. 12 is a diagram of a pipeline and analytical framework of a prediction/recommendation platform.

FIG. 13 is a flowchart of an example procedure to determine recommended action(s) for a group of users.

Like reference symbols in the various drawings indicate like elements.

### DESCRIPTION

Described herein is a data management platform to manage (including to store and transmit) large data records such as data records produced to handle complex data representations of neural signals. The example platform includes implementations (including hardware, software, and hybrid hardware/software implementations) directed to a framework to serialize, deserialize and store neural data (mostly time-series signals from sensors in neurotech devices) in an efficient, scalable, parallelizable, shardable, and space-saving way. The framework implements a protocol buffer for a language-neutral, platform-neutral, extensible mechanism for serializing structured neural data, and a time-series database optimized for time-stamped or time-series data such as neural signal data.

In the approaches described herein, a new data format, called Xneuro, is proposed to implement a unified neural data interface to facilitate scalable data import, standardization, search, and retrieval. The proposed Xneuro implementation was benchmarked across several high-throughput datasets, collected each in different modalities under various stimulus types and behavioral tasks, where traditional analytic pipelines find it difficult to collate. Experimentation and evaluation of the proposed implementations demonstrate the effectiveness and scalability of the framework by comparing these datasets to a series of cognitive models in a fast and scalable fashion.

The data management approaches described herein also include a service-based ecosystem for neurotech devices based on the concept of remote procedure call (RPC) in distributed computing. This system treats neural data pipelines as “Services” and is able to send messages between various servers and clients. This allows for both rapid storage and access to data at various frequencies, as well as from various sources and locations. This system distributes computing power, saving computational space and power and creates a full-stack ecosystem for emerging neurotech and wearable sensors. Finally, the system can streamline the sharing of resources and data among devices and/or companies to promote collaboration and greater insights from emerging neurotech devices. This resource sharing includes implementations that use individual brain computer interface client devices (whose main function is to collect neural and biometric data from individuals on which the devices have been deployed) to also be configured to act as servers that are able to perform dedicated processing services on behalf of other nodes in the neurotech network to which the brain interface client devices are connected. That is, one or more of the deployed neurotech devices can be configured to also act as a server able to receive request for service, and not only to transmit collected neural signal data to other nodes.

Also described herein is an example use application that uses the Xneuro framework and RPC-based neurotech network technology to implement a prediction/recommendation platform that processes neural signal data from multiple users to generate output (in this example, recommendation output). The technology thus implements a recommendation system for group decision making (e.g., shopping) that takes neural congruence into account. The system uses real-time signals from Brain-Computer Interface (BCI) devices and traditional user-based ratings to recommend an action that is most favored by the group (e.g., recommending an item that is most likely to be purchased by the group). The process itself combines the techniques of collaborative filtering, reinforcement learning, and session-based approaches. While the system was applied to shopping decisions, it can be used on a variety of applications needing more efficient and coordinated group decision-making, such as music, movies, food, and travel destinations, to name just a few examples.

Some additional examples of applications and use scenarios that rely on the technologies described herein include:
a) Healthcare: A patient with a neurological disorder such as epilepsy or Parkinson's disease wears a brain-computer interface (BCI) (data source, client) that records neural data in real-time (storing it locally by serializing it). This data is then transmitted (e.g., via RPC) to a remote server (database, deserializing the data, data source, server, client) for analysis and processing using messaging and remote procedure call (RPC) technology. The server can detect patterns in the data (generating new data, serializing intermediate data, storing in database) and provide feedback (via RPC) to the patient's healthcare provider (data source, database, server, client), who can adjust the patient's treatment plan (generating new data, serializing intermediate data, storing in database) as needed, and potentially transmitting new signals (via RPC) to the patient (database, client).
b) Game interface: controlling video game characters with the interpretation of neural data being performed at a game server. For example, a user wears a BCI (data source, client) that allows them to control a video game character using their thoughts. The neural data (after serializing it) is transmitted (via RPC) to a remote server (server, database, data source, which can deserializing it) using messaging and RPC technology, which processes the data and sends back instructions (via RPC, sending data which was serialized) to the user's device (client) on how to move the game character (another display unit, client, receiving commands, web interface). This allows for a more immersive gaming experience and could be used in virtual reality or augmented reality environments.
c) Education: an education interface in which neural data is interpreted at a server to generate personalized feedback. For example, a student wears a BCI (data source, client) that records their neural activity (storing it locally by serializing it) while they are learning a new skill, such as playing an instrument or speaking a foreign language (in some physical or virtual interfaces, client, server). The neural data is transmitted (via RPC) to a remote server (server, database, data source, deserializing) using the messaging and RPC technology, which processes the data and provides personalized feedback (as serializing messages) to the student on how to improve their performance. This could help students learn more effectively and efficiently.
d) Mental heath therapy feedback, for example, using neural data to assess a remote user's emotional state. For instance, a patient with depression wears a BCI (data source, client) that records their neural data (storing it locally by serializing it) while they are undergoing a cognitive-behavioral therapy (CBT) session. The neural data is transmitted (via RPC) to a remote server (server, database, data source, deserializing) using the messaging and RPC technology, which analyzes the data to determine the patient's emotional state and provides feedback (as serializing messages) to the therapist (client, database). This could help therapists tailor their interventions to each patient's individual needs and improve the effectiveness of therapy sessions.

Many other example use scenarios may likewise use the neural data processing and frameworks described herein.

### Xneuro Framework

As noted, a first aspect of the proposed data management approaches includes a framework to serialize, deserialize and store neural data (e.g., time-series signals from sensors in neurotech devices) in an efficient, scalable, parallelizable, shardable, and space-saving way. (Sharding is a process of splitting and storing a single logical dataset in multiple databases. By distributing the data among multiple machines, a cluster of database systems can store larger data sets and handle additional requests. Sharding may be necessary if a dataset is too large to be stored in a single database. Xneuro can support sharding innately, while H5 has to pre-specify it as a Sharded class first and then preprocess all data again in order to do sharing.

As neuroscience marches into the experimental era of a high-throughput, single-cell and real-time regime, the understanding of the nervous system is shifting from hypothesis-driven to data-driven modeling. Recent advances in machine learning and brain-computer interfaces have allowed the creation of mechanistic theories, prediction of neural signals, and utilization of this knowledge to create task-specific feedback loops for multiple purposes. The synergy between industrial and academic research is tighter now than ever, and thus, requires production-level treatment of data, which is scalable and efficient.

Neural data generated from neurotech devices such as those used by Neuralink and Fitbit are high dimensional, heterogenous, possess large digital footprints, and span multiple data types that are program-language specific and cross-incompatible. Furthermore, the cataloging of large volumes of data needs to be performed and stored in a fast, efficient, compact, and cost-effective way to allow for increasingly large neural model interfaces previously unachievable, improving user experiences in consumer-based neurotech, and providing platforms with a unique competitive advantage.

The proposed approaches of the Xneuro framework allow neural data to be serialized, deserialized, and/or stored in a manner that is efficient, scalable, and compact while providing flexibility for a wide range of workflows. Built on a uniquely designed protocol buffer and procedure, the Xneuro framework can read and write neural data faster and more compactly than traditional methods such as Hierarchical Data Format, Pickle, and Neurodata-without-borders in both a language- and platform-neutral way. Furthermore, the proposed framework has been optimized for time-series databases that are widely encountered throughout neural data collection, unlike current alternatives, and can be used to improve current data collection and storage systems while mediating cross-platform interactions that were previously inaccessible.

Current neural data collection systems lack speed, compactness, efficiency, and are limited by language- and platform-specific constraints. These challenges limit the size and complexity of feasible neural models and can impede user experiences especially in consumer applications of neurotech devices such as Fitbits. Meanwhile, storage of increasingly large databases such as time-series data becomes increasingly expensive. The Xneuro framework enables faster data collection and more compact data storage in a way that is language- and program-neutral, allowing for new cross-platform interactions. The technology can be employed by major neurotech companies, academics, and clinics to improve data collection, improve consumer products, and decrease costs associated with data storage.

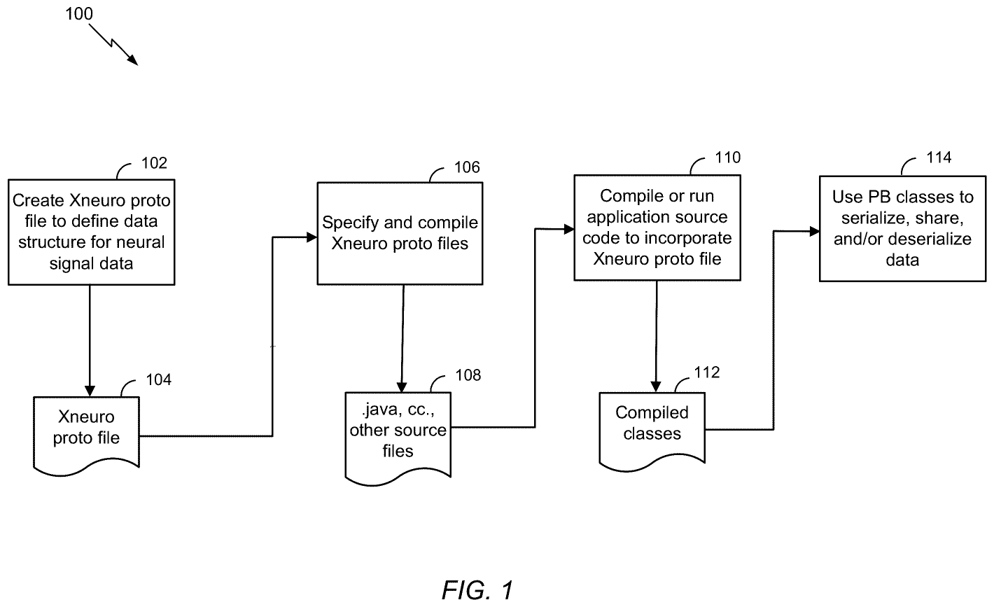

With reference to FIG. 1, a diagram of an example processing pipeline 100 to process data (such as neural data) is shown. The processing pipeline 100 facilitates two important features of embodiments of the proposed framework, namely, (1) presenting a unified format that stores different modalities of neural signals using protocol buffers (an open-source data format for serializing structured data to allow transmission between network devices in a platform-neutral way), and (2) optimized strategies to store time-series neural signals with time-series databases. With respect to feature (1), the protobuf mechanism allows for specifying a unified format that is workable across data modalities (e.g., EEG, fMRI, electrophysiology) by simply adding new specs on top of previous protos. This is advantageous because a company might grow and its products might introduce new sensors, for which it would be desirable to have data models that are extendable to the new formats with ease. Furthermore, neural data may be collected using in different devices, with different configurations, so that they cannot be processed or fused together easily (e.g., one user is using EEG device by company Y, and one user is using EEG or even fMRI by company X, with those different devices arranging and processing data using different formats that cannot match and be processed together).

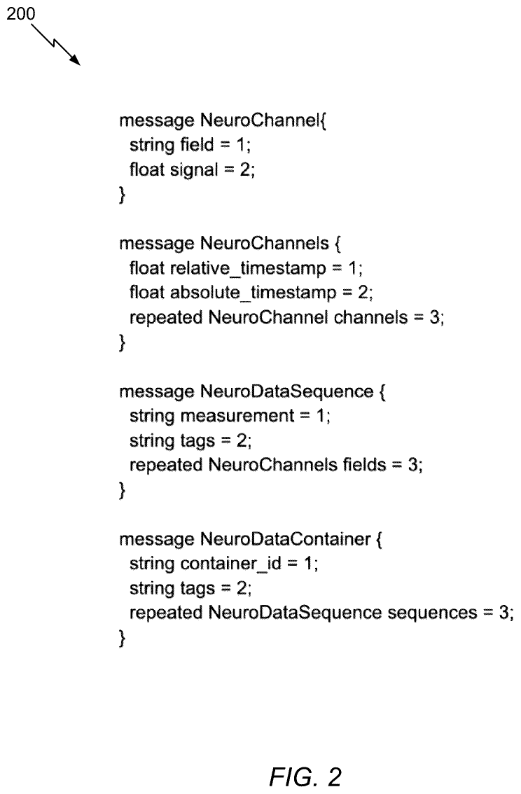

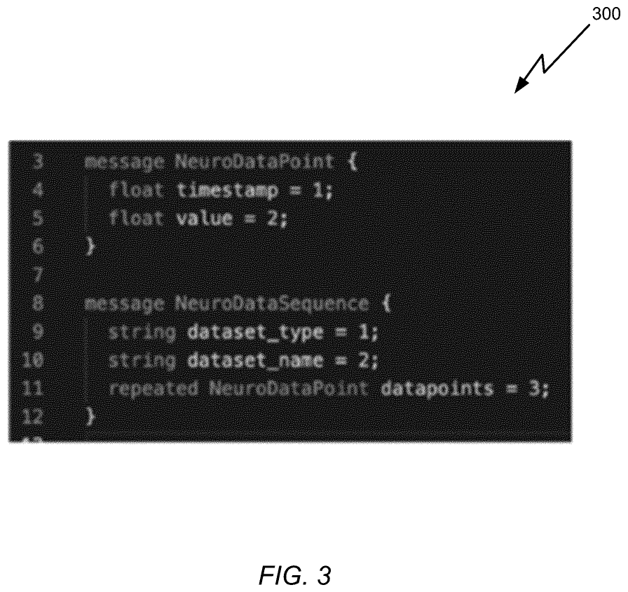

As depicted in FIG. 1, the processing pipeline procedure includes the following operations. First, the protocol buffer configuration(s) is specified (at block 102), resulting in a .proto file (at block 104). The protocol buffers provide a serialization format for packets of typed, structured data that are up to a few megabytes in size. The format is suitable for both ephemeral network traffic and long-term data storage. Protocol buffers can be extended with new information without invalidating existing data or requiring code to be updated. Protocol buffer messages and services are described by engineer-authored .proto files. Example Xneuro proto files 200 and 300 are shown in FIGS. 2 and 3, respectively. The two examples of proto file formats can be further extended to more complicated data formats. The example proto formats 200 and 210 are configured to store the values of the time-series samples as individual shardable messages, meaning that they can be easily resampled and analyzed in batches. Alternative proto files can specify the time points as a stream, and, in such cases, the processing will happen as soon as the pointer to a data sequence is accessed.

Turning back to FIG. 1, the proposed Xneuro framework specifies and complies, at block 106, the proto file(s) that defines the time-series data format for the neural signals. The framework ensures that these proto files are backward compatible, shardable, parallelizable, and able to process data in a stream-like fashion. According to the specific application(s) being used, a proto compiler is invoked at build time on the .proto files to generate resultant code in various programming languages (at illustrated in blocks 108 and 110) to manipulate the corresponding protocol buffer. The proto files specified for the Xneuro framework can be interpreted and compiled easily in different programming languages. This is particularly important in implementations that include sub-systems that use different languages/technologies. For instance, in some embodiments a web interface is used to monitor and visualize the neural state of a patient, in which case javascript may be needed to process the binary data files. However, in the same implementation, it may be desirable, at the same time, to compute an AI strategy in real-time to perform deep brain simulation, in which case a C++ application to access the data might be needed (due to the fast computing time achievable with C++ based applications). The Xneuro framework enables different processes (implemented using different technologies) to write, load and process the collected data (e.g., neural data) in a uniform way and with ease. For example, in some embodiments, the same protobuf can be used to power data usages by either a computer Python-based application running on a python server, and a mobile Swift-based application.

With continued reference to FIG. 1, each generated class (illustrated in block 112 of FIG. 1) contains simple accessors for each field and methods to serialize and parse the whole structure to and from raw bytes. The resultant protobuf classes are used for serializing, sharing (e.g., transmitting), and de-serializing data, as illustrated in block 114.

Thus, in some embodiments, a data management system is provided that includes one or more memory devices to store processor-executable instructions and neural data, and a processor-based controller, coupled to the one or more memory devices. The controller is configured, when executing the processor-executable instructions, to obtain one or more samples of neural data, and process the one or more samples of neural data according to protocol buffer definitions specifying formatting of neural data records for storage and transmission, to generate formatted neural data records. In some examples, a non-transitory computer readable media is provided that includes computer instructions executable on a processor-based device to obtain one or more samples of neural data, and process the one or more samples of neural data according to protocol buffer definitions specifying formatting of neural data records for storage and transmission, to generate formatted neural data records.

As noted, time-series optimization is an important aspect of the proposed Xneuro framework. Briefly, a time-series database (TSDB) is a database optimized for time-stamped or time series data. Time series data items are simply measurements or events that are tracked, monitored, downsampled, and aggregated over time. Time-series data items include, for example, server metrics, application performance monitoring, network data, sensor data, events, clicks, trades in a market, and many other types of analytics data. A time-series database is built specifically for handling metrics and events or measurements that are time-stamped. A TSDB is optimized for measuring change over time. Properties that make time series data very different than other data workloads are data lifecycle management, summarization, and large range scans of many records. In many industry applications (and especially web-based systems), time-series datasets are usually aggregated from the classical relational database. However, this is quite different from neuroscience data, or neural signals collected from neuro sensors. Neuro signals are usually collected as time-series measurements, making the serialization, deserialization, and storage of these data very different from existing ones. Time series databases systems are built around the predicate that they need to ingest data in a fast and efficient way. While traditional relational databases have a fast ingestion rate, from 20 k to 100 k rows per second. However, the ingestion is not constant over time. Relational databases have one key aspect that causes them to be slow when data tend to grow: indexes. Particularly, when new entries are added to a relational database, in embodiments where the table contains indexes, the database management system will repeatedly re-index the data so that it can later access it in a fast and efficient way. As a consequence, the performance of a DBMS tends to decrease over time. The load also increases over time, resulting in greater difficulties to access and read stored data. On the other hand, time-series databases are optimized for a fast ingestion rate. It means that such index systems are optimized to index data that are aggregated over time. As a consequence, the ingestion rate does not decrease over time and stays quite stable, around 50 k to 100 k lines per second on a single node. It is also to be noted neural data can be oscillatory, and, consequently, the time series can be optimized so to be stored in an optimized size, accounting for the oscillatory nature of the data, using some compression mechanism.

An important feature of many existing time-series databases (such as InfluxDB) is that they store the data in measurement sequences. The format of a time-series database is usually organized in measurement sequences of three fields, a measurement_name, a tag, and a value. This turns out to be highly effective in speeding up the ingesting of time-series data. As a result, proto files of the frameworks described herein are implemented based on this base structure. However, other alternatives to optimize access and retrieval of time-series data, such as tensor format, etc., may be used. Following execution of the processing pipeline 100, the example Xneuro proto file (such as the proto file 200 or 210) stores the time-series values as individual shardable messages (meaning that they can be resampled and analyzed in batches).

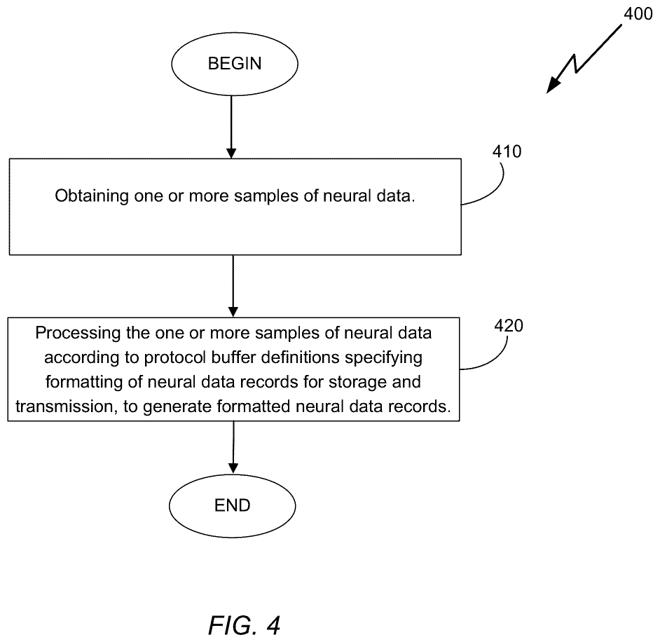

With reference next to FIG. 4, a flowchart of an example procedure 400 for management and processing of neural data (according to the Xneuro framework proposed herein, which is implemented using protocol buffers technology) is shown. The procedure 400 includes obtaining 410 one or more samples of neural data, and processing 420 the one or more samples of neural data according to protocol buffer definitions specifying formatting of neural data records for storage and transmission, to generate formatted neural data records. In various embodiments, processing the one or more samples of neural data according to the protocol buffer definitions may include arranging the one or more samples of neural data in timestamped measurement sequences comprising a measurement name field, a tag field, and a value field to hold a value derived from the one or more samples of the neural data.

In some examples, the procedure 400 may further include storing the formatted neural data records in a database. In such examples, storing the formatted neural data records in the database may include storing the formatted neural data records in a time-series database. In some embodiments, the procedure 400 may further include establishing communication links with network nodes of different, non-related, networks, with each of the networks being configured to execute respective different applications configured to process the formatted neural data records, and transmitting to at least one of the networks nodes of the different, non-related, networks one or more of the formatted neural data record for downstream processing. In such embodiments a first network, from the different, non-related networks, is implemented on a computing platform different from another computing platform implementing another of the different, non-related networks.

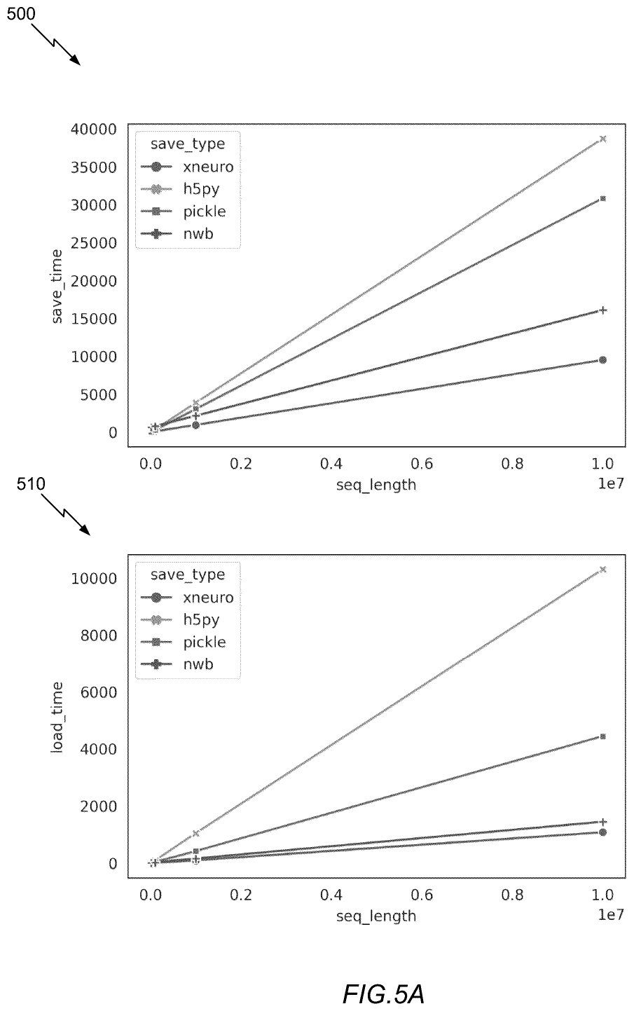

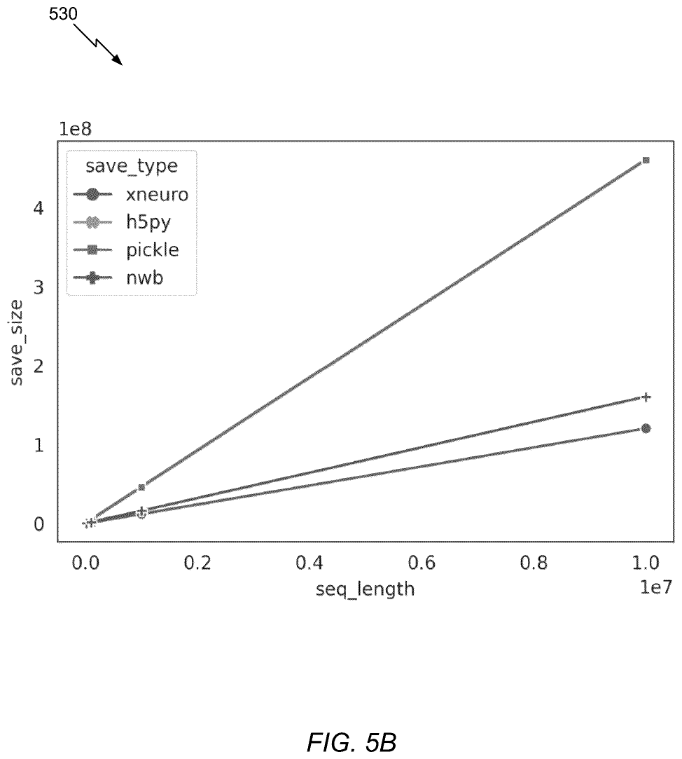

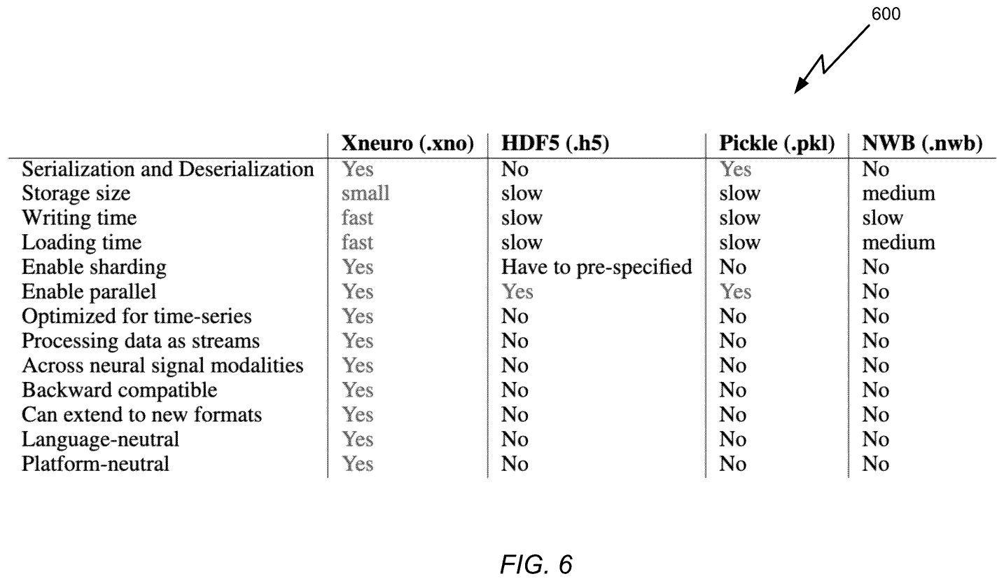

The Xneuro protobuf framework was tested and evaluated across several high-throughput datasets, collected each in different modalities under various stimulus types and behavioral tasks, where traditional analytic pipelines struggle to collate the data. The testing and evaluation of the Xneuro framework included comparing the performance of Xneuro to that achieved by various optimized (or near optimized) industry solutions that include: Hierarchical Data Format (H5), Pickle, and Neurodata-without-border (NWB). The comparison of these solutions against the Xneuro solution was conducted by writing and loading the voltage recording of a single neuron for 1 million time steps. FIG. 5A includes graphs 500 and 510 showing the time (ms) required to respectively save and load a neural signal of various lengths, under different storage types. FIG. 5B includes a graph 530 showing file size (bytes) required to save a neural signal of various lengths, under different storage types. Testing results showed that the Xneuro framework is 10×faster in speed, and requires 5×less memory storage than any of the other industry solutions. FIG. 6 includes a table 600 summarizing the performance comparison results of the Xneuro framework against the other tested industry solutions.

In evaluating and testing of the proposed Xneuro framework, the framework's compression ability (how much space it requires to store the same amount of data) was first evaluated. To store a time-series of neuronal recording data with 1 million timesteps, the h5, pickle and nwb require 45 MB, 44 MB and 15 MB, respectively, while Xneuro format only requires 11 MB. It is to be noted that one of the existing method, nwb, adopts a truncated format to reduce space, i.e., by shrinking the time-series to be an array. This already significantly reduces the storage requirements, but gives up the ability to perform stream-like processing and parallel computation. Because nwb uses a h5 format in its underlying code, if it were to adopt the same time-series representation as other benchmarks, the resultant data representation would require, or exceed, the 45 MB result achieved with h5. Thus, not only is the Xneuro framework the only method that enables (1) data stream processing, and (2) sharding (or parallel computing), but the Xneuro framework also stores the data in the most efficient lossless compression implementation, using the smallest amount of storage.

The next performance criterion to be investigated was the speed of the Xneuro framework relative to the other solutions considered. This performance criterion was evaluated by determining the time it took to write to a file with the same data format, and load it out as the same data format for in-session computation. In the writing case, h5 and pickle and nwb took at least 3 to 6 seconds to complete the task, while the Xneuro solution took around 1 sec to process the 1 million timesteps into the binary files. This is an important feature for neurotech products, because sensor data is usually recorded in real-time and is stored on the fly in fast iterations. If storage operations are too slow, then data cannot be stored in real-tine in time for downstream processing. In the loading case, the advantage is similar. Xneuro is faster than the other solutions investigated, and can be 10 times as fast as the h5 format. Moreover, neural data collections can come from noisy measurements (e.g., if a person uses am EEG headset as a commercial brain-computer interface (BCI) product, every time he or she wears it, it can be mapped slightly different to his brain, or when he or she is running, the signals can drift or collected in misaligned ways). Thus, noisy neural signals might require some fast in-device or on-server computation to align or deny them, and as such the data representations of the neural signals have to be stored and accessed fast and in minimal size. Traditional methods of managing neural data cannot handle it properly for real-time processing.

Accordingly, as described herein, the Xneuro technology is a framework to serialize, deserialize, and store neural data in a manner that is efficient, scalable, and compact while providing flexibility for a wide range of workflows. Built on a uniquely designed protocol buffer and procedure, this framework can read and write neural data faster and more compactly than traditional methods such as Hierarchical Data Format, Pickle, and Neurodata-without-borders in both a language- and platform-neutral way. The proposed solutions develop a unique protocol buffer and formatting framework that allows for specific cross-platform sharing of neural data that was previously unachievable, and allows for increasingly large and complex neural model inferences.

Furthermore, the Xneuro framework has been optimized for time-series databases that are widely encountered throughout neural data collection, and can be used to improve current data collection and storage systems while mediating cross-platform interactions that were previously inaccessible. Current neural data collection systems lack speed, compactness, efficiency, and are limited by language- and platform-specific constraints. These challenges limit the size and complexity of feasible neural models and can impede user experiences especially in consumer applications of neurotech devices such as Fitbits. Meanwhile, storage of increasingly large databases such as time-series data becomes increasingly expensive. The Xneuro framework proposed herein allows for faster data collection and more compact data storage in a way that is language- and program-neutral, allowing for new cross-platform interactions.

The Xneuro technology can be employed by major neurotech companies, academics, and clinics to improve data collection, improve consumer products, and decrease costs associated with data storage. The proposed framework can be used in various applications that require large volumes of data (e.g., neural data), including in some of the following user scenarios:
Brain-computer interface systems (e.g., interfaces developed and manufactured by Neuralink, Synchron, FitBit, Apple, etc.) all need to process and store large amount of biometric data comprising, for example, signals recorded from the neural sensors, wrists, hearts etc. The collected data needs to be stored in real-time (fast!) and compact (small!).
Historical neural data should also be accessed in real-time (fast!) for retrieval, or comparison (with user types, critical clinical profiles, or mood information etc.) These comparisons are likely in other devices programmed with different programming languages (cross-language, cross-platform).
Data sensed and collected from neural sensors needs to be extendable, scalable, and comparable to other sensors. For instance, Neuralink might develop future generation electrodes that have hundreds of probes. The framework proposed herein should be able to process the expected increased volume of data the same way that it might have processed data from Neuralink interface (for example, a Gen-1 interface is equipped with 50 probes). These analytical pipelines should be able to compare and analyze old data (backward-compatible, and extendable).
Two neurotech companies might want to collaborate together and share their data (e.g., Apple and Neuralink), and will thus need a data format that is unified across different measurement modalities (e.g., neural recordings in Neuralinks vs. ECG in Apple watch).
Digital health companies (Apple, FitBit, Amazon) might want to connect and interact with each other to operate on neural data they collected, and make the collected data compatible to other sales products they have. The proposed framework described herein allows this cross-platforms interactions in which large complex biometric data (such as neural data) can easily be shared and used (under a unifying format such as the Xneuro proposed herein) across a constellation of products (e.g., web-based products).
Storing biometric user data (including neural data) requires vast amounts of storage (on the order of terabytes (TB), or even petabytes) which costs money and uses up large amounts of energy. The proposed framework allows storage of such user data in a compact way, thus reducing storage and energy costs.
A user might have different neurotech devices belonging to the same company, and the same neural data recorded from a few sensors might be arriving at different rates. It would be advantageous to process the incoming data in parallel and in shards (parallelizable and shardable).

As noted, in some embodiments, key features of the proposed framework include, (1) the use of a unified format that stores different modalities of neural signals using protocol buffers, and (2) optimized strategies to store time-series neural signals with time-series databases. A user defines once how data is to be structured, and subsequently uses special generated source code to easily write and read structured data to and from a variety of data streams using a variety of languages. An approach based on protocol buffer can be suitable for processing and managing neuro data that, in neuroscience industry and labs, is still being stored as scientific data using inefficient formats. The proposed framework accommodates the temporal structure of the neural data, and enables the use of a specific type of proto configs in the protocol buffer to treat the data as sequences.

Neurotech developments that continue to rely on existing neuro-data data structures run a risk of being suboptimal, possibly because:
1) Most of the industrial applications (in large-scale web-based companies) are dealing with relational database type of data, and that is quite different from what the data would be in the neuroscience industries, which are full of high-dimensional time-series data; and
2) Most existing neurotech startups have not reached a point where their neuro sensors require web communications. However, given the pace at which this technological field is developing, new applications will increasingly require more compact and faster communication of neuro-data. In any event, using large-scale web-based technologies for (the currently seemingly smaller-scale) neural data processing, already offers a significant benefit upon existing methods.

### Communication Framework for Transmitting Neural Data Between Client and Server Nodes

Having defined the Xneuro data structures used to efficiently represent and format neural signal data, a second framework in support of managing and processing neural data is the communication network that is used to transmit neural data between various network nodes (e.g., different neurotech devices) to implement an efficient platform to manage and process neural data. Thus, described herein are systems, devices, method, and other implementations for messaging and processing neural data between servers and clients using remote procedure calls. The proposed framework treats different neural data processing pipelines as services and sends neural signals and preprocessed intermediates as messages between different servers and clients. This becomes a working example of a service-based neurotech system.

The neurotech industry is a growing business. However, moving from the lab to a profitable product is a hard task. Data transmission implementation in the context of neuroscience research and in the context of a commercial product are entirely different problems presenting disparate challenges. In neuroscience research, or a lab, there is generally no need to transmit data from multiple places to multiple places. Rather, data (e.g., neural signal data) is usually stored locally at one server location. In a research lab setting typically one individual is working on one dataset, without other individual accessing or modifying the same files. On the other hand, in a commercial setting (involving neurotech products), other storage and transmission requirements need to be addressed. Commercial manufacturers and service providers typically involve large-scale web-based or service-based applications, involving different teams generating different user data or intermediate metric data. These data items are usually generated from different sources across the globe from potentially billions of users and millions of sensors or data collectors. Such data items are not only written asynchronously at irregular frequencies (bursts), but are also accessed by different teams for different reasons and at different irregular frequencies. Furthermore, users often only want to access a certain entry, or a block of entry, from a certain data stream.

The proposed approach described herein presents a framework for a web-based ecology of neurotech devices. Treating different neural signal processing pipelines as different web-based services enables a much large-scale business models across different product lines. The proposed approaches thus provide a framework to treat different neural data processing pipelines as services, and sends neural signals and preprocessed intermediates as messages between different servers and clients. This becomes a working example of a service-based neurotech system. For instance, some possible user scenarios for in which the proposed framework can be implemented include:
The brain-computer interface and wearable companies (e.g., Neuralink, Synchron, FitBit and Apple) collect in real-time high-dimensional signals from various neural sensors on their brains, wrists, hearts etc. This data needs to be sent to the server in real-time, and is generally received intermittently from users and/or other nodes in irregular timed and sized batches.
Real-time stream data will need to be merged with the historical neural data of a particular user or user cohorts, and be accessible to other processing pipelines.
There would likely be different services, or teams that need to access these data in trunks. For instance, one function/team wants to retrieve user A's real-time neural recordings to compare with the recordings for other users and classify user A's recordings into certain clinical profiles. Another function/team may wish to access the EEG recording profiles of a group of NYC users in age 20-30 to output seasonal trends in NYC area.
Data intermediates form different Services can be combined together into bigger Service pipelines. For instance, one conglomerate might want to recommend certain products on sale to certain user, given the preprocessed emotional profile of a certain user (computed based on a data stream produced by sensors of his/her wearable device such as an Apple Watch).
A neurotech company might want to sell its neural sensor API to several companies, and these buyer companies need to access the API of the neural databases of the neurotech company in irregular frequencies and sometimes the same time.
Different digital health companies might want to connect/link neural data they collected to other sales products they or their partner companies have.

In some embodiments, the proposed neurotech network and messaging framework is based on the remote procedure call. In distributed computing, a remote procedure call (RPC) is a process by which a local computer application causes a procedure (subroutine) at a remote device/node, with a different address space than that defined for the initiator device, to be triggered and executed (commonly on another computer on a shared network). The triggering call is coded as if it were a normal (local) procedure call, without the programmer explicitly coding the details for the remote interaction. That is, the programmer produces the same code whether the subroutine is local to the executing program, or remote. This is a form of client— server interaction (caller is client, while the executing device is the server), typically implemented via a request—response message passing system.

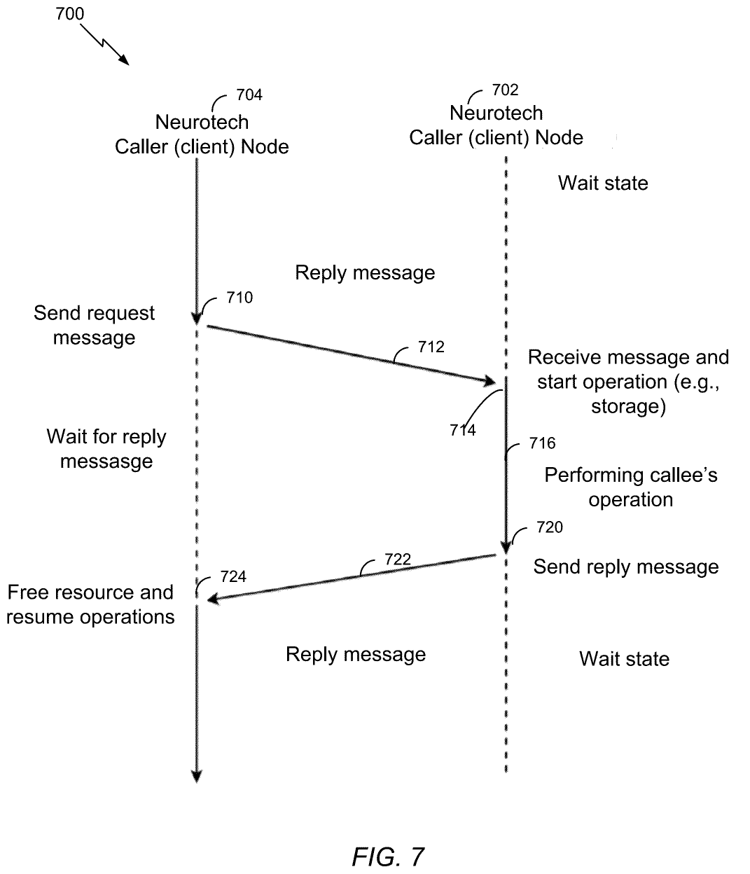

One way to implement this type of network communication and/or messaging system is to place the remote procedure call system in the Operating System. For example, consider a situation where an operating system for a neurotech device (referred to as NeurOS) is to be realized. In such an implementation, a messaging system allowing communication between remote neurotech devices could be realized that would allow an initiating device to process local data at a remote neurotech device that support a particular procedure that is to be applied to the local data at the initiating device. With reference to FIG. 7, a flow diagram 700 depicting an example messaging procedure to trigger/invoke a remote neurotech procedure is shown. As shown, a neurotech server process 702 is configured to wait and monitor for incoming request messages from one or more callers (client processes) such as neurotech client process 704. It is to be noted that the neurotech client process may have a dual role of both requesting services from other remote nodes in the network (e.g., to process data, such as neural signal data, stored at the client process 704), and executing its own particular neurotech process to respond to requests (for the particular neurotech process) arriving from other nodes in the network. Suppose the neurotech caller 704 is a neurotech device comprising a sensor. Upon receipt of data measured by the sensor (as a result of a schedule collection of data, or due to controlled or uncontrolled activation of the sensor), the neurotech caller collects data, such as neural signal data. Assume that in this scenario the neurotech caller device does not have sufficient storage to store the data, and thus the neurotech caller 704 performs a call procedure, at point 710, that causes a message request, that includes at least some of the collected data (e.g., collected neural data that may have been formatted according to the Xneuro protocol buffer framework discussed above) and procedure parameters needed to configure and control the server process at the neurotech callee node/device. The request message (marked as message 712) is sent to the neurotech callee 702, which upon receiving, at 714, the request message 712 (e.g., requesting storage of the data included in the message 712) begins (optionally after performing an authentication procedure, e.g., based on a private-public key pair procedure, to confirm the validity of the request message) to perform the processing the neurotech callee has been configured to perform (in this case to store the received data, for example, in a time-series database such as those discussed above in relation to the Xneuro framework). Having received the message (indicating that there is sensor data to be recorded), and optionally having authenticated the message, the procedure (subroutine) performed by the neurotech callee node records (stores) the data included with the request message 712 (during an interval 716). In some embodiments, the receipt of the message 712 will cause the neurotech callee node 702 to switch from a ‘wait’ state to an ‘on’ state, thus allowing preservation of power during periods at which there are no data items to be processed.

After the neurotech callee's procedure is performed, the callee procedure is configured to transmit to the neurotech caller node 704 a reply message 722 that contains results produced by execution of the procedure at the neurotech callee node 704. In situations where the procedure performed by the callee is a storage procedure, the reply message 722 may include confirmation that the storage operation was performed, and may optionally include information relating to the storage operation (e.g., a record number or address to indicate the specific storage location where the data sent by the caller node 704 has been stored). Upon receipt of the message 722, and confirmation that the storage operation (in the example of FIG. 7) has been performed (and thus a re-sending of the data by the caller node 704, to either the callee node 702 or some other node in the distributed network that can perform the storage operation, is not needed) the caller node can free any held-up resources to perform the next task (e.g., send the next neural data item, etc.) It is to be noted that while the caller (client process) node and callee (server process) node are depicted in FIG. 7 as separate nodes, the caller and callee processes may be executed, in some embodiments, on the same device or node, or alternatively, more than one remote callee can be contacted to perform parts of the request indicated in the message 712. It is also to be noted that multiple servers and clients can be located in different locations and can host different data. Each client can send different requests to different servers without interfering with any client's or server's operation, and conversely each server can also act as a client and send requests for data processing to other nodes in the neurotech network (e.g., when that node does not have the resources or is not configured to perform some task for which it needs to send a request to another node). Thus, the neurotech network of the proposed framework described herein is configured to execute multiple RPC sessions concurrently.

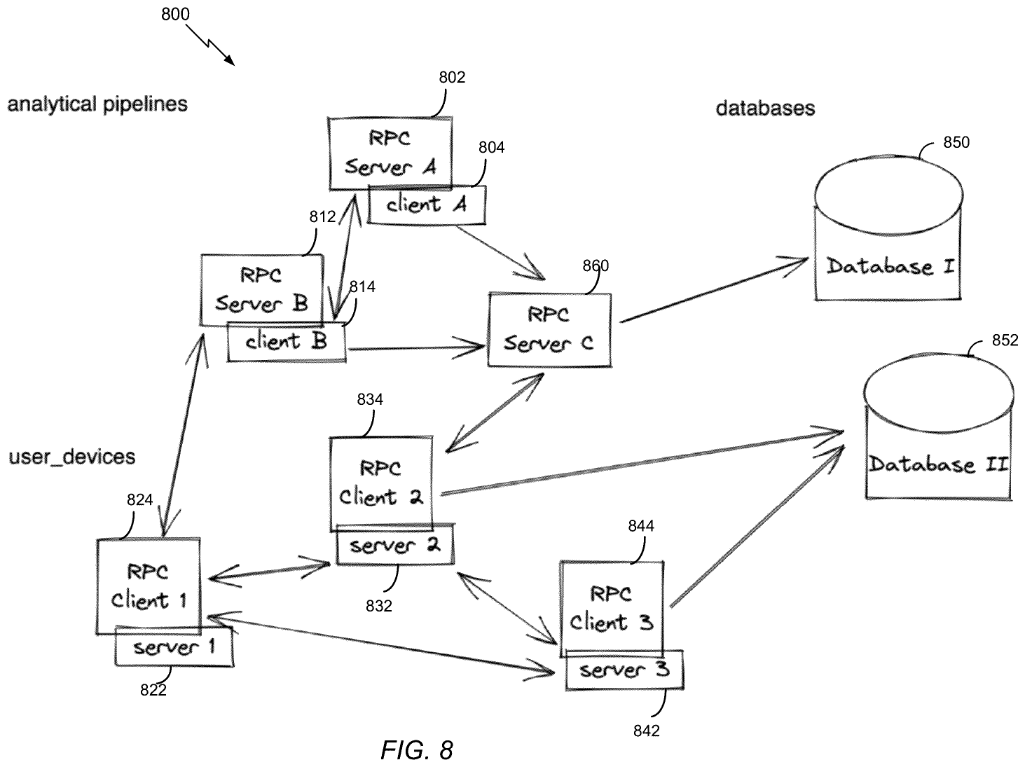

With reference now to FIG. 8, a schematic diagram of an example neurotech RPC-based network 800 is shown. The network 800 includes user devices (e.g., neurotech sensors to procure neural signal measurements from users) that are also configured to function as RPC server that provide designated processing services responsive to RPC requests received from other neurotech devices or from other servers that are part of the network 800. The service-based neurotech system/network 800 includes, in this example, two databases 850 and 852 (which may be part of two servers dedicated to managing stored data). The example network 800 also includes three user devices (e.g., neurotech sensors to procure neural signal measurements from users) that are numbered as RPC clients 1, 2, and 3 (and are marked with reference numerals 824, 834, and 844, respectively). In some examples, the user devices can be configured both as clients that send data processing requests (for data collected by the devices, or for data received from other devices), and as servers (comprising companion server modules 822, 832, and 842) that provide services responding to request from the user devices or from any other interconnected node of the network 800. While the user devices are shown as able to function as both RPC clients and RPC servers, in some examples, one or more of the user devices may be configured to only act as clients capable of collecting neural data and sending requests to interconnected servers of the network 800. Under those circumstances, client-only devices would be configured to send data and requests (commands, requests for data formatting and configurations, etc.) and data for processing at any servers or databases interconnected to the clients (such as the server 812 directly connected to the client device 824, or the database 852 interconnected to the client device 844).

In some examples, one or more nodes of the network 800 can include server-only nodes that may be used for data analysis (e.g., they form part of the analytical pipeline defined in the network 800), for data processing (reconfiguring or reformatting the data, or transforming the data into a different representations), or for any other functions required to process and analyze the data collected by client nodes (whether such clients nodes are part of a dual-role computing device that can also act as a server, or whether such nodes are neurotech client devices). When a server (such as the server 860) of the network 800 is part of the analytical pipeline of the network 800, the server may be configured to provide downstream analysis for data that was processed upstream (e.g., by any combination of the upstream clients, whether they are server-based clients or neurotech client devices capable of collecting and transmitting data, and/or applying some processing to their collected data or data originating at another device). The downstream node can thus accept input data from upstream nodes and produce analytic output (e.g., control signals to actuate devices, labels or embedding vectors when the downstream server performs machine learning processes, etc.) Thus, in some embodiments, RPC pipelines can be combined, with, for example, one analytical pipeline (say, the server 860 (Server C)) acting as a downstream analysis pipeline that takes inputs from Servers A and B (marked as servers 802 and 812), and output an analytical output, which is sent to the Database I (marked as database 850) for storage. It is to be noted that regardless of the specific services that servers are configured to render, interaction between the various nodes is done via the transmission of requests and data, with the requests generally including procedure calls (e.g., according to Remote Procedure Call protocol). This type of arrangement can significantly save storage space and computing resources.

Operationally, there are different frameworks that can implement an RPC system. For instance, gRPC is a modern open source high performance Remote Procedure Call (RPC) framework that can run in any environment. It can efficiently connect services in and across data centers with pluggable support for load balancing, tracing, health checking and authentication. It is also applicable in “last-mile” processing for distributed computing to connect devices, mobile applications, and browsers to backend services. As will be discussed below in greater detail, an example embodiment that was used for testing and evaluation was implemented using a gRPC framework, although other RPC-type frameworks could have been used. In gRPC, a client application can directly call a method on a server application on a different machine as if it were a local object, making it easier to create distributed applications and services. As in many RPC systems, gRPC is based around the idea of defining a service, and specifying the methods that can be called remotely with their parameters and return types. On the server side, the server implements this interface and runs a gRPC server to handle client calls. On the client side, the client may have a stub module configured to perform necessary conversions and transformations of parameters when sending an RPC request to a remote node that operates on a different computing platform than that of the client. Under a gRPC-based platform clients and servers can run and talk to each other in a variety of environments—from remote servers to users' desktop applications—any of which can be implemented in any gRPC supported language. So, for example, a gRPC server can implemented be in Java and communicate with clients in Go, Python, or Ruby. In addition, the latest Google APIs may have gRPC versions of their interfaces, letting users easily build Google functionality into their applications.

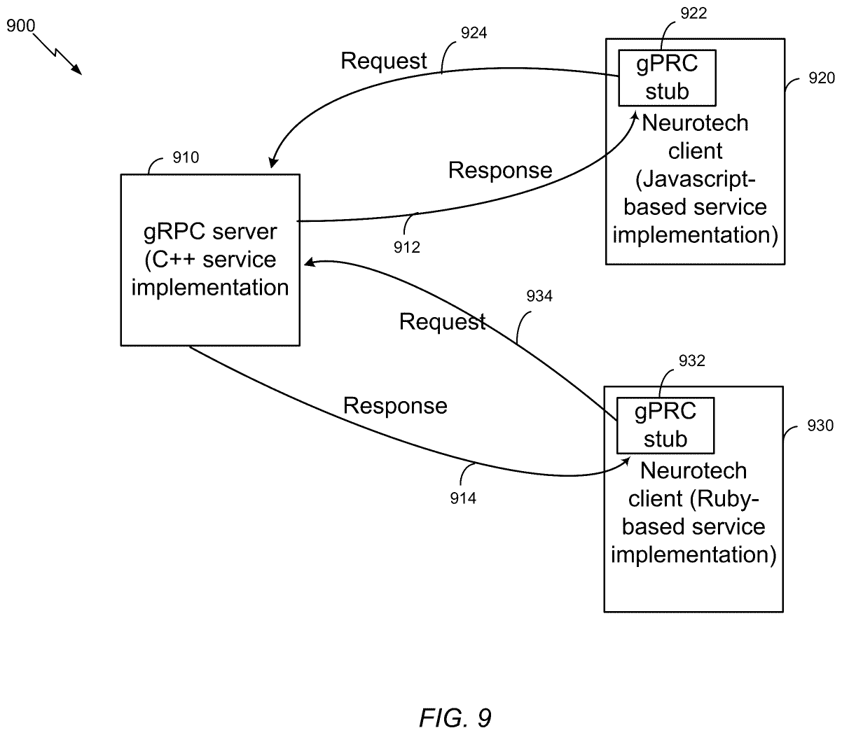

FIG. 9 provides an example of a gPRC-based network 900 that includes a gPRC server 910, configured to provide a service implemented through C++, in communication with two clients 920 and 930 that may each be neurotech user devices that collect neural data from respective users on which the client devices 920 and 930 are deployed. In the example of FIG. 9, the clients 920 and 930 are implemented as dedicated clients without providing any services to interconnected nodes of the network 900 (that is, they act as clients-only, without being configured to act as servers). However, as described herein, in some embodiments, either or both of the clients 920 and 930 may be configured to include server modules that can receive data from other network nodes, process the data (using an implementation based on the native computing characteristics of the client devices), and communicate the resultant output (be it processed data or some analytical output) to the initiating node or to some other node.

As further illustrated in FIG. 9, each of the clients 920 and 930 includes a gPRC stub 922 and 932, respectively, that is configured to transform requests generated according to the local computing environment of the respective device to a format compatible with the destination of the message. Thus, for example, a request 924, comprising data and a message indicating a processing request to be performed by the server 910 (e.g., to reformat neural data included in the request 922, and store the data in a local database) may have been generated by the gPRC stub 922 to convert the Ruby-based message (generated according to the native computing implementation of the client 920) to a format compatible with the C++-based computing environment of the gPRC server 910. The converted request 924 may include any parameters and control signaling needed to properly launch the service available at the server 910. It is to be noted that the message sent to the server 910 (or any of the messages depicted in FIG. 9) may be configured as protobuf messages generated, for example, according to the approaches discussed herein in relation to FIGS. 1-6. Upon completion of the service requested by the client 920 to be performed at the server 910, the server 910 sends a response 912 (which may include data output, or a confirmation that the service has been performed), and the gPRC stub 922 may convert the response 912 into data (e.g., neural data) and control data (e.g., procedure calls, signaling data, etc.) that conforms to the Ruby-based client implementation of the client 920.

The example network 900 illustrates the advantages of the proposed platform. Different neurotech devices requiring real-time analysis (of the data those devices collected) can use the computing resources of more powerful nodes (such as the server 910 which is capable of running C++-based applications that potentially can improve the performance speed relative to the computing platforms available at the client devices).

In another example, a visualization dashboard (not specifically shown in FIG. 9) to provide visualization and statistics services may be implemented using Javascript on another server (or a client server, if the client 930 were to be configured to act as a server that performs the visualization dashboard functionality), and a Python-based server is used to provide analytics. In this example, a client device may send two requests to the Python-based analytics server, with a first request asking the server to generate an electrophysiological recording dataset given certain configurations, and with a second request asking the server to compute several useful statistics of the said generated neural signals (in this example, one server is configured to provide several different services, which may correspond to different applications running on the same service). The Javascript Client then receives a resultant message from the Python-based server containing these useful statistics in a human-readable summary text, and presents the statistics on the visualization dashboard.

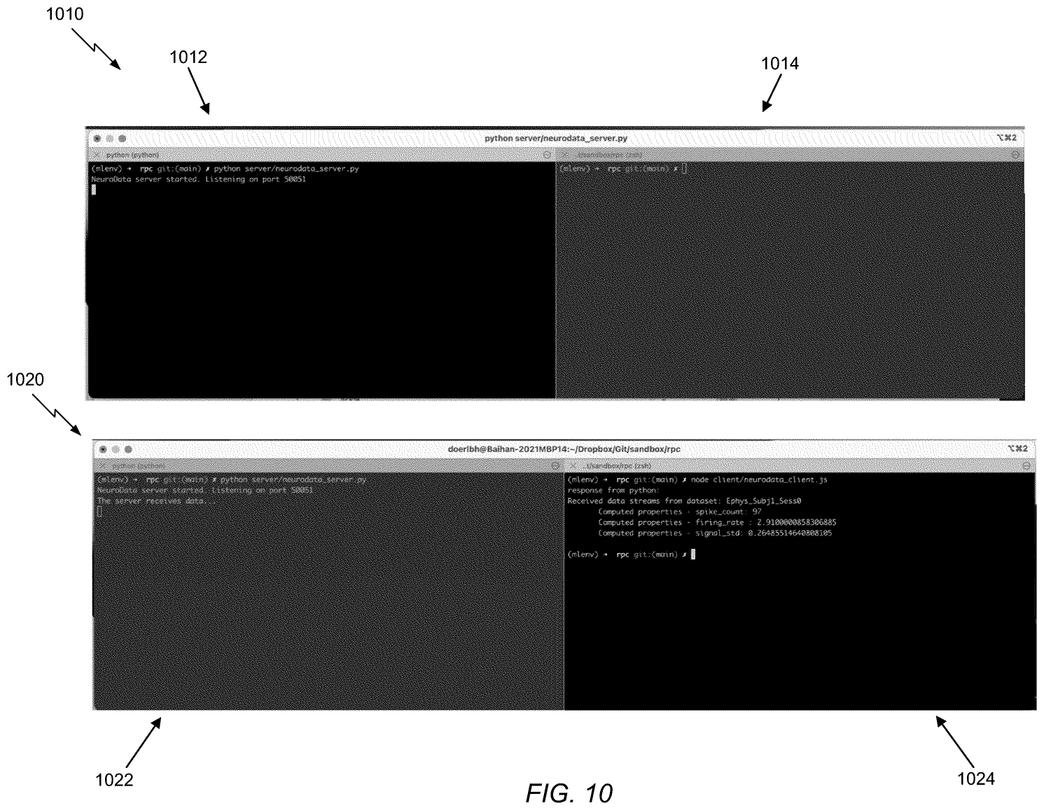

With reference next to FIG. 10, screenshots 1010 and 1020 of a working example of an RPC-based communication between a client and a server in a neurotech network (such as the ones illustrated in FIGS. 8 and 9) are shown. The top screenshot 1010 shows a left panel 1012 of a terminal instance of the server end. As described herein, a server node of a neurotech network can host large databases (to store copious amount of data, such as neural signal data from multiple users), as well as perform heavy computations for different “services” (forming part of one or more analytical pipelines). The right panel 1014 of the screenshot 1010 is a terminal instance on a client end (e.g., a client node, implemented using a particular neurotech device that is configured m in part, to sense neural signals of an individual). In this working example, a user (or the client device autonomously) can send a request to the server end (e.g., the client end can send a data configuration information, a dataset with data representing, for example, a neural signal sample, information about the pipeline configuration to inform what response the client device is expecting, etc.) As illustrated in the left panel 1012, the server, at the particular instance captured by the screenshot, is in listening mode, and waiting for a request to service (either form the client associated with the right panel 1014, or with any other client or server node of the network).

The screenshot 1020 of FIG. 10 captures a later instance of the interactive session between the client node associated with the right panel 1014 and the server associated with the panel 1012. Particularly, the JavaScript-code implemented client sends, as shown in the right panel 1024, two requests. The first request creates a dataset (e.g., uploading a dataset) and sends it to the server. The second request asks the server to process the freshly uploaded electrophysiology dataset by computing the number of spikes in the neural signals, the firing rate of the neurons, and the standard deviation (i.e., noise) of the time-series. The screenshot 1020 illustrates that the server receives the dataset and requests, computes in real-time the results, and sends result data back to the client (for display on the client terminal, as shown in the panel 1024) as a response printout.

Thus, in some embodiments, a neurotech communication system is provided that includes multiple network devices comprising at least a first network device and a second network device (e.g., a neurotech device that collects neural signals), with each of the multiple network devices including one or more memory devices to store processor-executable instructions and neural signal data, and a processor-based controller coupled to the one or more memory devices. The processor-based controller of the first network device is configured, when executing associated processor-executable instructions, to receive at the first network device, from the second network device, a remote procedure call (RPC) message comprising a first data representation of neural signal data obtained by the second network device and servicing data specifying parameters to cause execution of a first servicing procedure executable on the first network device, perform the first servicing procedure to process the first data representation of the neural signal data to generate result data, and transmit, by the first remote network device, another RPC message to a destination network device, the other RPC message including the result data.

In some additional embodiments, a non-transitory computer readable media is provided that includes computer instructions executable on one or more processor-based devices to receive at a first network device, from a remote, second network device, a remote procedure call (RPC) message comprising a first data representation of neural signal data obtained by the second network device and servicing data specifying parameters to cause execution of a first servicing procedure executable on the first network device, perform the first servicing procedure to process the first data representation of the neural signal data to generate result data, and transmit, by the first remote network device, another RPC message to a destination network device, the other RPC message including the result data.

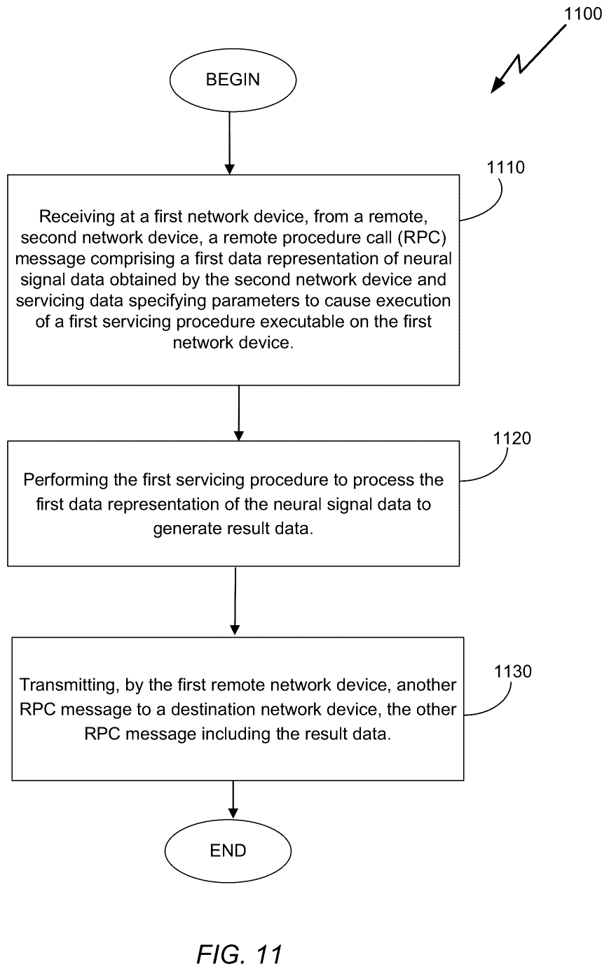

With reference next to FIG. 11, a flowchart of an example procedure 1100 for processing and communicating neural signal data is shown. The procedure 1100 includes receiving 1110 at a first network device (e.g., a server device, such as the servers 802, 812, and 860 depicted in FIG. 8), from a remote, second network device (a neurotech device, that collects neural signal data from individuals), a remote procedure call (RPC) message comprising a first data representation of neural signal data obtained by the second network device and servicing data specifying parameters to cause execution of a first servicing procedure executable on the first network device. The procedure 1100 further includes performing 1120 the first servicing procedure to process the first data representation of the neural signal data to generate result data, and transmitting 1130, by the first remote network device, another RPC message to a destination network device, the other RPC message including the result data.

In various examples, the result data may include resultant processed neural signal data, and in such examples the method may further include storing, at a database (e.g., the databases 850 and 852 of FIG. 8) coupled to the destination network device, the resultant processed neural data. In some embodiments, the first data representation of the neural signal data may be generated according to protocol buffer definitions specifying formatting of neural signal data samples for storage and transmission.

In some implementations, the destination network device may be the second network device. In such implementations, transmitting the other RPC message may include transmitting the other RPC message to the second network device for further processing, at the second network device, the result data generated by the first remote network device. The second network device (e.g., the neurotech device) may implement a different servicing procedure than the servicing procedure executing on the first network device (in order to take advantage of the resources of the second network device), and the procedure 1100 may further include receiving by the second network device one or more request messages, from network devices in communication with the second network device, requesting performance of the different servicing procedure executable by the second network device, processing with the different servicing procedure the one or more received request messages to generate respective result data, and transmitting RPC reply messages responsive to the one or more RPC requests. In such embodiments, each of the first servicing procedure, executable on the first network device, and the different servicing procedure, executable on the second network device, may be implemented as one or more of, for example, an algorithmic analytical procedure executed in response a received RPC request message, and/or a machine-learning model to generate predictive data responsive to the RPC request message.

In some examples, the other RPC message may further include another servicing data specifying other parameters to cause execution of a second servicing procedure, different from the first servicing procedure executable at the first remote network device, to process the result data generated in response to the RPC message from the second network device. The first servicing procedure executable on the first remote network device may be implemented on a computing platform different than the computing platform on which the second servicing procedure, executable on the destination network device, is implemented. The RPC message may be generated using an RPC stub module implemented at the second network device to conform with computing environment characteristics of the first network device. The RPC stub may be configured to generate the RPC message to conform with any of a plurality of different computing platforms of respective multiple network devices forming, together with the first network device, a neurotechnology network to collect and process neural signals measured from one or more users. In some embodiments, the second network device may include a neurotechnology device configured to interface with the brain of a user.

Thus, the technology described herein is directed to a service-based ecosystem for neurotech devices based on the concept of remote procedure call in distributed computing. This system treats neural data pipelines as “Services” and is able to send messages between various servers and clients. This allows for both rapid storage and access to data at various frequencies, as well as from various sources and locations. The proposed framework distributes computing power, saving on computational space and power and creates a full-stack ecosystem for emerging neurotech and wearable sensors. The proposed framework can streamline the sharing of resources and data among devices and/or companies to promote collaboration and greater insights from emerging neurotech devices. The technology describes a web-based or service-based system for neurotech devices based on RPC used in distributed computing, and introduces a framework to treat neural data processing pipelines as Services and send both unprocessed and preprocessed signals as messages between different servers and clients. The technology also allows for fast, irregular storage of data from different sources and locations, allows for streamlined, irregular access to data from different sources, supports connection to other data processing pipelines, combine sub-pipelines into pipelines, able to run multiple pipelines simultaneously, and uses gRPC (a commonly used framework for RPC).

Example user scenarios in which the neurotech networking approach described herein may be used include:
Healthcare: A patient with a neurological disorder such as epilepsy or Parkinson's disease wears a brain-computer interface (BCI) that records neural data in real-time. This data is then transmitted to a remote server for analysis and processing using the messaging and remote procedure call (RPC) technology. The server can detect patterns in the data and provide feedback to the patient's healthcare provider, who can adjust the patient's treatment plan as needed.
Gaming: A user wears a BCI that allows them to control a video game character using their thoughts. The neural data is transmitted to a remote server using messaging and RPC technology, which processes the data and sends back instructions to the user's device on how to move the game character. This allows for a more immersive gaming experience and could be used in virtual reality or augmented reality environments.
Education: A student wears a BCI that records their neural activity while they are learning a new skill, such as playing an instrument or speaking a foreign language. The neural data is transmitted to a remote server using the messaging and RPC technology, which processes the data and provides personalized feedback to the student on how to improve their performance. This could help students learn more effectively and efficiently.
Mental health: A patient with depression wears a BCI that records their neural data while they are undergoing a cognitive-behavioral therapy (CBT) session. The neural data is transmitted to a remote server using messaging and RPC technology described herein, which analyzes the data to determine the patient's emotional state and provide feedback to the therapist. This could help therapists tailor their interventions to each patient's individual needs and improve the effectiveness of therapy sessions.
Web services interface to allow effective communication of brain signals between users (optionally with some intermediate computations), where, as discussed above, each node can act as both a client and a server.

In summary, the technology described herein could enable a wide range of applications in fields such as healthcare, gaming, education, and mental health by allowing for the real-time processing and analysis of neural signal data.

Another example user scenario in which neural data can be processed and managed through the neurotech RPC approach and/or the Xneuro protocol buffer approach is discussed in greater detail below in the section relating to prediction and recommendation operations based on neural signals in social settings.

Below is a table summarizing the performance of the service-based neurotech framework in comparison to traditional lab-based approach:

| | | |
|---|---|---|
| | Service-based | Traditional lab-based |
| Features | neurotech framework | neurotech approach |

| | | | |
|---|---|---|---|
| Storing | Frequency | Many times, Irregular | Once |
| the data | Source | From different sources | From one person |
| | | | (source) |
| | Location | Into different | Into one location |
| | | locations (parallel | (no redundancy |
| | | into different servers, | protection) |
| | | redundancy protection) | |
| | Speed | Fast, only when | Slow, always need to |
| | | required | preprocess everything |
| | Extendibility | Easily just add a data | Have to process |
| | | in without a fuss | everything again |
| Accessing | Frequency | Many times, Irregular | Sporadic |
| the data | Source | From different sources | Can be multiple |
| | | | accesses, but can be |
| | | | conflicting with one |
| | | | another |
| | Access | Streamlined, no | Messy, can risk |
| | management | contamination | overwriting the data |
| Pipelines | Connection to | Easily extendable and | Not supported |
| | other pipelines | innately supported | |
| | Combining | Innately supported | Not supported |
| | Sub-pipelines | | |
| | into pipelines | | |
| | Multiplicity | Different pipelines | Not supported |
| | | can happen at the same | |
| | | time without conflicts | |

Considering different neural signal processing pipelines as different web-based services enables implementations of large-scale business models across different product lines. It is imagined that it would be highly important for any neuroscience product to be able to extend to multiple platforms across multiple Services and multiple data sources.

### Prediction and Recommendation Operations Based on Neural Signals in Social Setting

As noted, another user example in which the Xneuro and the neurotech service network frameworks can be used to manage neural signals from multiple users is to support prediction and recommendation applications. Humans are social animals. While most recommendation systems assume a single-user preference prediction, real-world decision-making often involves a preference unit of a group, such as families, couples, or other groups of individuals. For instance, in close relationships like a romantic relationship, people often prioritize the interest of their significant others over themselves and prefer to make important decisions together. These secondary sociopsychological factors of consumer preference can be hard to characterize. As an emerging class of interaction paradigm, the brain-computer interface (BCI) includes a sensor to extract brain signals, a computer to analyze them, and a downstream task or device to relay the message for a desired action or goal.

Disclosed herein is a proposed new paradigm of brain-computer recommendation system in group settings that takes into account the congruence of neural signals. Through a web-based application, mobile-end interfaces, and brain measurement devices (e.g., neurotech devices, arranged in a distributed network configuration such as the ones discussed herein), the framework shows that recommendation/prediction systems can be boosted by augmented neural interfaces and social engagements.

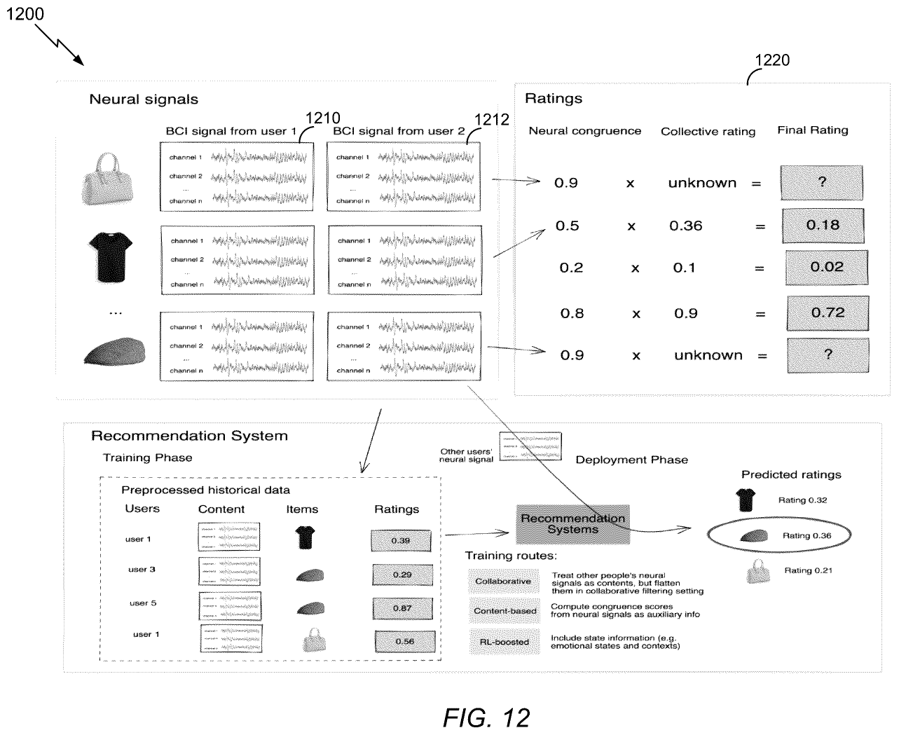

With reference to FIG. 12, a diagram of a pipeline and analytical framework of a prediction/recommendation platform 1200 (the platform is referred to as “BrainCart” when used to make retail recommendations) is shown. To assist with the discussion of the intricacies of the platform, a few terms are first defined. In the context of the example retail recommendation platform 1200 (it will be appreciated that the platform can be used in other commercial situations, and for non-commercial applications), the “items” the system recommends are products with their price tags, the “users” are the subjects or participants of this collective shopping experience, the “contents” are the neural signals measured in real time from the brain computer interfaces each of the subjects wears, and, lastly, the “ratings” would be weighted collective ratings derived from a formulation that takes into account the congruence of neural signals. A database to store historical data relating to the above-defined terms can be used to train (initially, or intermittently after the system has become operational) the recommendation platform 1200.

The analytical framework proposed herein implements a special collective rating metric/score that measures the possibility that a collective decision (e.g., a purchase decision) can be made. This weighted collective rating may be a product of two quantities. The first one is the congruence among the neural signals of the subjects when they are thinking about a product item. This can be any similarity measure, such as cosine similarity between the neural signals of two or more subjects (referred to as the neural congruence measure/score). A collective rating (statistical rating), which is an aggregation of all explicit ratings made by users (e.g., the current users or previous shoppers) with respect to particular items is then obtained (e.g., an on-line rating for some consumer product). If no user had previously reacted to a certain product (e.g., an item on a shopping application does not have an associated rating compiled from users' inputs), the collective neural-based rating quantity is unknown. If one or more users had written a rating to this product, the statistical rating (e.g., mean) compiled from those users' specified ratings can be used to represent the collective statistical rating. The final rating would simply be, in some embodiments, the collected rating weighted by the neural congruence score. In other words, the more congruent the neural signals of a collective purchase group are, the closer the final rating is towards the collective rating. And the smaller the neural congruence level, the more down-weighted the rating is (up to a value of zero). Other ways to combine available statistical ratings for a product(s) with neural congruence level(s) determined from multiple users for the product(s) may be used.

Thus, as illustrated in FIG. 12, the platform 1200 receives neural signal data from multiple user (2 users in the example of FIG. 12), which may comprise of n signals collected from each user in response to particular stimuli (e.g., in response to the visual of a handbag, n channels of signals are collected from each of the two users to provide neural signal sets 1210 and 1212). The neural signal sets are provided to a rating unit 1220 that derives a composite rating score for the stimuli items (i.e., the items that resulted in the particular neural signals collected by neurotech devices) based on the collected neural signals and, in some embodiments, previous rating associated with the item (e.g., rating based on explicit feedback, for example in the form of online reviews). The composite rating can be computed according to different formulations and methodologies. In the example illustrated in FIG. 12, the composite rating is derived by first determining neural congruency between the users' neural signals, and combing the determined congruency score with a pre-determined collective rating of the item under consideration. While the composite rating used in FIG. 12 derives the final rating as a product of the congruence score and the collective rating, the two values can be combined using other functions or formulations (e.g., averaging the two scores, applying some non-linear operator to the two values, etc.) As noted, the neural congruence can be determined by determining the similarity between neural signals (e.g., through cosine similarity) of the signals. Since, in the example of FIG. 12, neural signals from n channels are collected, the neural score can be the average of the cosine similarity computed separately for each channel, or alternatively, the overall similarity of the neural signals can be computed through other formulations. In some embodiments, the cosine similarity may be computed from a simplified representation of the neural signals (e.g., by sampling and normalizing the signals, by applying a machine learning transform model to the neural signal to produce vector representations, etc.) Having computed the composite rating (final rating) for a particular item, the particular item may be recommended (to one of the users whose neural signals are being measured, or to some other unrelated user) if the composite rating exceeds some pre-determined rating threshold. Alternatively, a decision to recommend a particular item may be determined using a machine learning engine that accepts as input the final rating for the item and/or additional data points (further information about the users, contextual information related to the particular item, and so on).

Implementations of the rating unit, or any other module of the platform 1200, may be realized using multiple interconnected client neurotech devices (e.g., interconnected wirelessly) that can be configured to act as companion servers of the client neurotech devices to perform processing services for signals collected by any of the interconnected devices. For example, neural signal processing (e.g., sampling and/or vectorizing it) may be performed by one or more designated neurotech devices, congruence determination may be determined by one or more other neurotech devices, and computing the final rating can be performed by yet a further one or more neurotech devices. The neurotech devices may communicate with other devices in the interconnected network through the transmission of RPC requests (as described herein in relation to FIGS. 7-11) that include data that may have been formatted according to the Xneuro framework described herein. As discussed herein, in some embodiments, the neurotech devices may be interconnected to a server that can perform at least some of the functionality of platform 1200.

Since the users, items, contents, and ratings have all been defined, the recommendation engine can be easily crafted with content-based and collaborative filtering. As a first step, item-based collaborative filtering is used as the recommendation engine. Since session turns are generally sequential and can specify a state or timestamp, during the training stage reinforcement learning and session-based approaches can be used to improve the recommendation-making operations, which can be neuroscience or psychiatry-inspired to provide better characterization of neural signals and interpretable insights. For the special rating formulation proposed herein, the recommendation system is more likely to recommend products which are both (1) high ratings across the subjects and (2) eliciting similar neural profiles among the collective purchase group (for other rating formulation, other factors might impact the nature of the recommendations made). As a result, it is intuitive to believe that a group is more likely to make a collective purchase because the recommended product activates the participants' brains in similarly positive ways.

Implementations of the proposed prediction/recommendation framework described herein were tested and evaluated. In particular, an interactive multi-user web-based recommendation platform called “BrainCart” was implemented and evaluated. First, a group of participants (in this evaluation, two) was each equipped with a brain-computer interface device. In this example, two OpenBCI handband kits were utilized, with each measuring eight (8) channels of Electroencephalography (EEG) signals in real-time. Both 8-channel signal streams were fed into a laptop with a Python program that registers the neural profiles. Then, all the participants viewed the web application on a shared screen, which included an image of a product and its price tag. To avoid having unaccounted effect of the price in purchase decision, prices were randomized to be uniformly distributed between $50 to $100 (most of the products that were shown were clothing articles in that price range). Then, each participant inputted their preferences on their cell phone, where they each opened a web page to log their user ID's and transmit their responses back to the server with a web socket. These mobile screens were independent from one another, and the users could choose their ratings from 1 to 5 (with 5 being the best), and indicate whether or not they would like to buy this product. The participants were not able to see the other user's ratings or choices. If all participants chose “Buy,” a collective purchase was made. The goal of the platform described herein is to recommend the next product that was most likely to lead to a collective purchase.

After a set number of iterations (e.g., 100 products), the shared computer screen reported how many objects were purchased, and the individual mobile screen showed how many products that the respective user wanted to buy but ended up not able to buy due to a disagreement in the group. Since for the purpose of the evaluation it was desired to have the demonstration system be lightweight, a pre-training process was not included, but the recommendation system was allowed to recompute its ranking at every iteration (that all subjects respond to a priced product). As a result, the recommendation system gradually stabilized after at least 20 to 30 rounds. For better performance, a pre-registration process could be included where the users can calibrate their preferences by viewing many products in advance. The system may refresh all its parameters at the end of each session to fit new data.

Thus, in various examples, a system is provided that includes multiple brain-computer interface devices to obtain from multiple users neural signals relating to an item, and one or more processor-based controllers, in communication with the brain-computer interface devices. The one or more processor-based controllers are configured to obtain a pre-determined user rating for the item, derive a collective neural-signal-based rating for the item based on the pre-determined user rating and the neural signals from the multiple users, and perform an item-related operation based on the collective neural-signal-based rating for the item. In additional examples, a non-transitory computer readable media is provided that includes computer instructions executable on one or more processor-based devices to obtain from multiple users neural signals relating to an item, obtain a pre-determined user rating for the item, derive a collective neural-signal-based rating for the item based on the pre-determined user rating and the neural signals from the multiple users, and perform an item-related operation based on the collective neural-signal-based rating for the item.

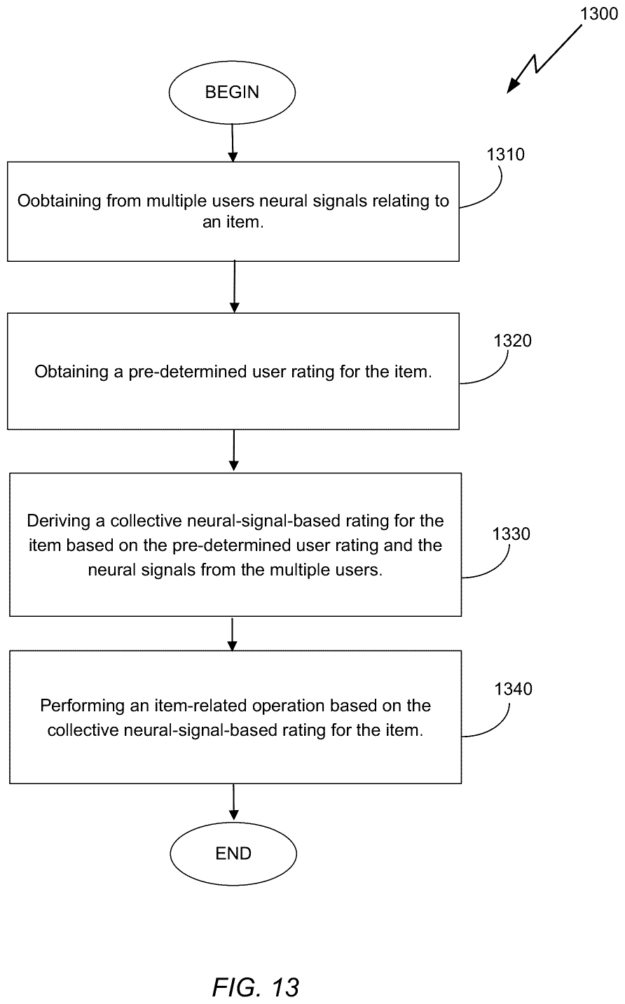

With reference now to FIG. 13, a flowchart of an example procedure 1300 to determine recommended action for a group of users is shown. The procedure 1300 includes obtaining 1310 from multiple users neural signals relating to an item, obtaining 1320 a pre-determined user rating for the item, deriving 1330 a collective neural-signal-based rating for the item based on the pre-determined user rating and the neural signals from the multiple users, and performing 1340 an item-related operation based on the collective neural-signal-based rating for the item.

In some embodiments, deriving the collective neural-signal-based rating may include determining a neural congruence level of the neural signals for the multiple users, and weighing the pre-determined user rating by the neural congruence level. In such embodiments, determining the neural congruence level may include computing similarity level between data representations of respective neural signals for two or more of the multiple users. In some examples, performing an item-related operation may include generating a purchase recommendation for a consumer product. Obtaining neural signals may include measuring neural signals for respective ones of the multiple users with multiple neurotech brain interface devices interconnected to a neurotech network. At least one of the multiple neurotech brain interface devices may be configured to perform operations on data collected by other of the multiple neurotech brain interface devices in response to RPC request transmitted from the other of the multiple neurotech brain interface devices.

Accordingly, the proposed technology relates to a group recommendation system that takes neural congruence into account. The goal is to recommend an action or an item (e.g., a product to purchase) that will most likely be desired by the group. The proposed system can be applied to other applications requiring group decisions such as music, movies, food, and travel destinations. However, it is not straightforward to directly plug in off-the-shelf recommendation systems to brain signals. Instead, the proposed framework takes into account the innate complexity of the activity (e.g., purchase behaviors) where the decision is often a joint decision made by a group of people. By analyzing the neural signals in group settings, recommendation systems can potentially be more advantageous than traditional systems. The proposed system is based on the assumption that if the brain signals sync among users, the behavior of a particular activity (e.g., buying or not buying a similar rated item) is more consistent and more likely than the case where their brain signals do not match. This assumption is supported by recent neuroscience findings. In the demonstration system that was evaluated, the system was able to recommend priced products that were most likely agreed and liked by all participants. As noted, determination of overall agreement (congruence) between users is achieved by obtaining multi-channel neural signals (alternatively, other biometrics, such as those obtained by smart watches, fMRI imaging data, or other kinds of signals indicating mind set of a user may be used instead of or in addition to neural signals), and preprocessing of multi-channel data. The collected neural signal data (or other types of user data indicative of the mind set) are pooled and their congruence computed (e.g., using a similarity criterion, such as cosine similarity) as a common metric to compare neural signals.

Recommendation systems can be utilized in many ways. For instance, a music recommendation system (which works well with wearing a brain computer interface device or VR glasses), a movie recommendation system, a dinner recommendation system, a travel destination recommendation system, or a recommendation system for any activity or endeavor involving decision-making, may be implemented in a manner similar to the implementations discussed for FIGS. 12 and 13. Taking into account what other people are “thinking” can make group decisions faster and more effective.

### Additional Embodiments

Performing the various techniques and operations described herein may be facilitated by a controller device (e.g., a processor-based computing device). Such a controller device may include a processor-based device such as a computing device, and so forth, that typically includes a central processor unit or a processing core. The device may also include one or more dedicated learning machines (e.g., neural networks) that may be part of the CPU or processing core. In addition to the CPU, the system includes main memory, cache memory and bus interface circuits. The controller device may include a mass storage element, such as a hard drive (solid state hard drive, or other types of hard drive), or flash drive associated with the computer system. The controller device may further include a keyboard, or keypad, or some other user input interface, and a monitor, e.g., an LCD (liquid crystal display) monitor, that may be placed where a user can access them.

The controller device is configured to facilitate, for example, processing, managing, and utilizing neural signal data. The storage device may thus include a computer program product that when executed on the controller device (which, as noted, may be a processor-based device) causes the processor-based device to perform operations to facilitate the implementation of procedures and operations described herein. The controller device may further include peripheral devices to enable input/output functionality. Such peripheral devices may include, for example, flash drive (e.g., a removable flash drive), or a network connection (e.g., implemented using a USB port and/or a wireless transceiver), for downloading related content to the connected system. Such peripheral devices may also be used for downloading software containing computer instructions to enable general operation of the respective system/device. Alternatively and/or additionally, in some embodiments, special purpose logic circuitry, e.g., an FPGA (field programmable gate array), an ASIC (application-specific integrated circuit), a DSP processor, a graphics processing unit (GPU), application processing unit (APU), etc., may be used in the implementations of the controller device. Other modules that may be included with the controller device may include a user interface to provide or receive input and output data. The controller device may include an operating system.

In implementations based on learning machines, different types of learning architectures, configurations, and/or implementation approaches may be used. Examples of learning machines include neural networks, including convolutional neural network (CNN), feed-forward neural networks, recurrent neural networks (RNN), etc. Feed-forward networks include one or more layers of nodes (“neurons” or “learning elements”) with connections to one or more portions of the input data. In a feedforward network, the connectivity of the inputs and layers of nodes is such that input data and intermediate data propagate in a forward direction towards the network's output. There are typically no feedback loops or cycles in the configuration/structure of the feed-forward network. Convolutional layers allow a network to efficiently learn features by applying the same learned transformation(s) to subsections of the data. Other examples of learning engine approaches/architectures that may be used include generating an auto-encoder and using a dense layer of the network to correlate with probability for a future event through a support vector machine, constructing a regression or classification neural network model that indicates a specific output from data (based on training reflective of correlation between similar records and the output that is to be identified), etc.

The neural networks (and other network configurations and implementations for realizing the various procedures and operations described herein) can be implemented on any computing platform, including computing platforms that include one or more microprocessors, microcontrollers, and/or digital signal processors that provide processing functionality, as well as other computation and control functionality. The computing platform can include one or more CPU's, one or more graphics processing units (GPU's, such as NVIDIA GPU's, which can be programmed according to, for example, a CUDA C platform), and may also include special purpose logic circuitry, e.g., an FPGA (field programmable gate array), an ASIC (application-specific integrated circuit), a DSP processor, an accelerated processing unit (APU), an application processor, customized dedicated circuitry, etc., to implement, at least in part, the processes and functionality for the neural network, processes, and methods described herein. The computing platforms used to implement the neural networks typically also include memory for storing data and software instructions for executing programmed functionality within the device. Generally speaking, a computer accessible storage medium may include any non-transitory storage media accessible by a computer during use to provide instructions and/or data to the computer. For example, a computer accessible storage medium may include storage media such as magnetic or optical disks and semiconductor (solid-state) memories, DRAM, SRAM, etc.

The various learning processes implemented through use of the neural networks described herein may be configured or programmed using TensorFlow (an open-source software library used for machine learning applications such as neural networks). Other programming platforms that can be employed include keras (an open-source neural network library) building blocks, NumPy (an open-source programming library useful for realizing modules to process arrays) building blocks, etc.

Computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal.

In some embodiments, any suitable computer readable media can be used for storing instructions for performing the processes/operations/procedures described herein. For example, in some embodiments computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as flash memory, electrically programmable read only memory (EPROM), electrically erasable programmable read only Memory (EEPROM), etc.), any suitable media that is not fleeting or not devoid of any semblance of permanence during transmission, and/or any suitable tangible media. As another example, transitory computer readable media can include signals on networks, in wires, conductors, optical fibers, circuits, any suitable media that is fleeting and devoid of any semblance of permanence during transmission, and/or any suitable intangible media.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

What is claimed is:

1. A method for management of neural data, the method comprising:
obtaining one or more samples of neural data; and
processing the one or more samples of neural data according to protocol buffer definitions specifying formatting of neural data records for storage and transmission, to generate formatted neural data records.

2. The method of claim 1, further comprising:
storing the formatted neural data records in a database.

3. The method of claim 2, wherein storing the formatted neural data records in a database comprises:
storing the formatted neural data records in a time-series database.

4. The method of claim 1, wherein processing the one or more samples of neural data according to the protocol buffer definitions comprises:
arranging the one or more samples of neural data in timestamped measurement sequences comprising a measurement_name field, a tag field, and a value field to hold a value derived from the one or more samples of the neural data.

5. The method of claim 1, further comprising:
establishing communication links with network nodes of different, non-related, networks, wherein each of the networks is configured to execute respective different applications configured to process the formatted neural data records; and
transmitting to at least one of the networks nodes of the different, non-related, networks one or more of the formatted neural data record for downstream processing.

6. The method of claim 5, wherein a first network from the different, non-related networks is implemented on a computing platform different from another computing platform implementing another of the different, non-related networks.

7. A method for processing and communicating neural signal data, the method comprising:
receiving at a first network device, from a remote, second network device, a remote procedure call (RPC) message comprising a first data representation of neural signal data obtained by the second network device and servicing data specifying parameters to cause execution of a first servicing procedure executable on the first network device;
performing the first servicing procedure to process the first data representation of the neural signal data to generate result data; and
transmitting, by the first remote network device, another RPC message to a destination network device, the other RPC message including the result data.

8. The method of claim 7, wherein the result data includes resultant processed neural signal data, and wherein the method further comprises:
storing, at a database coupled to the destination network device, the resultant processed neural data.

9. The method of claim 7, wherein the first data representation of the neural signal data is generated according to protocol buffer definitions specifying formatting of neural signal data samples for storage and transmission.

10. The method of claim 7, wherein the destination network device is the second network device, and wherein transmitting the other RPC message comprises transmitting the other RPC message to the second network device for further processing, at the second network device, the result data generated by the first remote network device.

11. The method of claim 7, wherein the other RPC message further comprises another servicing data specifying other parameters to cause execution of a second servicing procedure, different from the first servicing procedure executable at the first remote network device, to process the result data generated in response to the RPC message from the second network device.

12. The method of claim 7, wherein the first servicing procedure executable on the first remote network device is implemented on a computing platform different than the computing platform on which the second servicing procedure, executable on the destination network device, is implemented.

13. The method of claim 7, wherein the RPC message is generated using an RPC stub module implemented at the second network device to conform with computing environment characteristics of the first network device, wherein the RPC stub is configured to generate the RPC message to conform with any of a plurality of different computing platforms of respective multiple network devices forming, together with the first network device, a neurotechnology network to collect and process neural signals measured from one or more users.

14. The method of claim 7, wherein the second network device comprises a neurotechnology device configured to interface with a brain of a user.

15. The method of claim 7, wherein the second network device implements a different servicing procedure than the servicing procedure executing on the first network device, and wherein the method further comprises:
Receiving by the second network device one or more request messages, from network devices in communication with the second network device, requesting performance of the different servicing procedure executable by the second network device;
processing with the different servicing procedure the one or more received requests to generate respective result data; and
transmitting RPC reply messages responsive to the one or more RPC requests.

16. The method of claim 15, wherein each of the first servicing procedure, executable on the first network device, and the different servicing procedure, executable on the second network device, is implemented as one or more of: an algorithmic analytical procedure executed in response a received RPC request message, or a machine-learning model to generate predictive data responsive to the RPC request message.

17. A method comprising:
obtaining from multiple users neural signals relating to an item;
obtaining a pre-determined user rating for the item;
deriving a collective neural-signal-based rating for the item based on the pre-determined user rating and the neural signals from the multiple users; and
performing an item-related operation based on the collective neural-signal-based rating for the item.

18. The method of claim 17, wherein deriving the collective neural-signal-based rating comprising:
determining a neural congruence level of the neural signals for the multiple users; and
weighing the pre-determined user rating by the neural congruence level.

19. The method of claim 18, wherein determining the neural congruence level comprises:
computing similarity level between data representations of respective neural signals for two or more of the multiple users.

20. The method of claim 17, wherein performing an item-related operation comprises:
generating a purchase recommendation for a consumer product.

21. The method of claim 17, wherein obtaining neural signals comprises:
measuring neural signals for respective ones of the multiple users with multiple neurotech brain interface devices interconnected to a neurotech network.

22. The method of claim 21, where at least one of the multiple neurotech brain interface devices is configured to perform operations on data collected by other of the multiple neurotech brain interface devices in response to RPC request transmitted from the other of the multiple neurotech brain interface devices
